(19) United States
(12) Patent Application Publication
(10) Pub. No.: US 2013/0180541 A1
(43) Pub. Date: Jul. 18, 2013

(54) WATER SOLUBLE HAIR GLUE

(71) Applicant: Cynthia Lorraine Bobinac, Battle Creek, MI (US)

(72) Inventor: Cynthia Lorraine Bobinac, Battle Creek, MI (US)

(21) Appl. No.: 13/733,365

(22) Filed: Jan. 3, 2013

Related U.S. Application Data
(60) Provisional application No. 61/632,075, filed on Jan. 18, 2012.

(51) Int. Cl. A61K 8/65; A61Q 5/06; A61K 8/34

(52) U.S. Cl. CPC A61K 8/65; A61K 8/345; A61Q 5/06
USPC 132/203; 424/70.14; 141/11

(57) ABSTRACT

A water soluble hair glue is designed as a temporary adhesive to adhere to human or animal hair. The water soluble hair glue is an excellent alternative to bobby pins or rubber bands and can secure even short and fine hair in place. The water soluble hair glue can also be used to add hair extensions, beads, glitter, bows, flowers and other embellishments. The water soluble hair glue is designed to dissolve and rinse away when washing the hair with shampoo and warm water.

### CROSS-REFERENCE TO RELATED APPLICATION

This application claims the benefit of priority of U.S. provisional application No. 61/632,075, filed Jan. 18, 2011, the contents of which are herein incorporated by reference.

### BACKGROUND OF THE INVENTION

The present invention relates to hair glues and, more particularly, to a water soluble hair glue that does not require harsh, solvent-based removers to clean and remove the hair glue from the user.

Keeping hair in place with bobby pins, rubber bands, clips, or barrettes is a problem, as these devices can pull, fall out, break hair and do not work on short or fine hair.

Hair glue is available and is often used to hold hair extensions in place. Conventional extension glue, however, is not water soluble and must be removed with harsh, solvent-based removers.

Moreover, one's options are even more limited when looking to attach accessories to a pet's fur, as it is often too short for clipping into the fur, and, when clips are possible, they are often quite visible. Conventional extension glue is not meant for an animal's fur and can prove difficult to remove.

As can be seen, there is a need for an improved hair glue that is easy to apply and water soluble for easy removal and clean-up.

### SUMMARY OF THE INVENTION

In one aspect of the present invention, a water soluble hair glue comprises glycerin; gelatin; and water, wherein the water soluble hair glue is a solid at room temperature and can be warmed to a liquid state for application to a user's hair.

In another aspect of the present invention, a method for preparing a water soluble hair glue comprises mixing glycerin and gelatin; warming in a microwave oven for about 30 seconds; allowing the mixture to cool; warming in the microwave oven for about 40 seconds; adding water to the resulting mixture while warm; and allowing the resulting mixture to cool to a solid state.

In a further aspect of the present invention, a method for styling a user's hair with either a hair style held in place by a water soluble hair glue or with an embellishment held in place with the water soluble hair glue comprises warming a water soluble glue to a liquid state; applying the water soluble glue to the user's hair, the water soluble hair glue including glycerin, gelatin, and water, wherein the water soluble hair glue is a solid at room temperature and can be warmed to a liquid state for application to a user's hair; attaching a portion of the user's hair to the water soluble glue, or attaching the embellishment to the water soluble glue, while the water soluble glue is still in the liquid state; and allowing the water soluble glue to cool to hold the portion of hair or the embellishment in place.

These and other features, aspects and advantages of the present invention will become better understood with reference to the following drawings, description and claims.

### BRIEF DESCRIPTION OF THE DRAWINGS

FIG. 1 is a flow chart describing a process for making a water soluble hair glue for storage in a container until ready for use, according to an exemplary embodiment of the present invention;

FIG. 2 is a flow chart describing a process for preparing the storage container made in the process of FIG. 1 for use and using it to glue an object, according to an exemplary embodiment of the present invention;

FIG. 3 is a perspective view showing a water soluble hair glue of the present invention being applied to a user;

FIG. 4 is a perspective view of an object being attached to a user's hair where the water soluble hair glue was applied as shown in FIG. 3;

FIG. 5 is a detailed side view of a portion of FIG. 4, showing the application of the object to the user's hair with the water soluble hair glue;

FIG. 6 is a perspective view showing application of the water soluble hair glue of the present invention for hair styling; and

FIG. 7 is a perspective view showing an exemplary hair style using the water soluble hair glue of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a water soluble hair glue that is designed as a temporary adhesive to adhere to human or animal hair. The water soluble hair glue is an excellent alternative to bobby pins or rubber bands and can secure even short and fine hair in place. The water soluble hair glue can also be used to add hair extensions, beads, glitter, bows, flowers and other embellishments. The water soluble hair glue is designed to dissolve and rinse away when washing the hair with shampoo and warm water.

The water soluble hair glue of the present invention can allow a person to create several different hair styles and add endless embellishments to human and animal hair. Users will no longer have to suffer with bobby pins or rubber bands breaking or coming off. The water soluble hair glue of the present invention is easy to use, easy to apply and easy to remove when a hair style change or embellishment removal is desired.

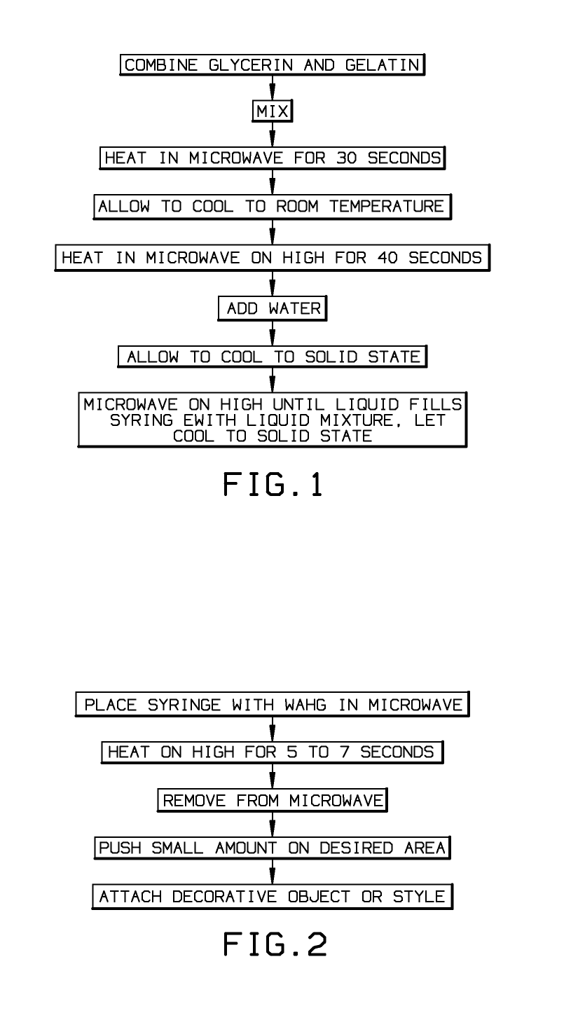

Referring to FIG. 1, to make the water soluble hair glue of the present invention, a user would first combine glycerin and gelatin, mix them together, and warm in a microwave oven (such as a typical residential microwave oven operating at from about 500 to about 1300 Watts, for example) for about 30 seconds. The resulting mixture is allowed to cool to room temperature and is heated in the microwave oven on high for about 40 seconds. Typically, the microwave can warm the mixture to a temperature from about 123° F. to about 165° F. Water can be added while the mixture is still hot and the resulting mixture is allowed to cool to a solid state. The solid is then warmed in the microwave oven again until liquid, allowing one or more applicators to be filled. The applicators can be allowed to cool and stored until ready for use. Typically, the applicators can be a plastic syringe, for example. These steps could be performed, for example, in a manufacturing facility and the filled applicators can be packaged for sale.

The above preparation and times are designed for mixing about 20 ml glycerin, 10 ml gelatin and about ¼ teaspoon of water. The specific volumetric ratio of glycerin:genatin:water can vary and are typically in the range of 17-23:8-12:0.8 to 1.5, often in the ratio of 20:10:1.2. These measurements can be scaled, as desired, with the appropriate scaling of heating and cooling times.

Referring to FIG. 2, when a user desires to use one of the applicators filled by the above method (or purchases such a pre-filled and pre-prepared applicator), the user can simply place the applicator in the microwave and heat on high for 5 to 7 seconds—just long enough to create a warmed liquid in the applicator. The user can then apply an appropriate amount to a desired area and style their hair accordingly or attach a desired object or embellishment.

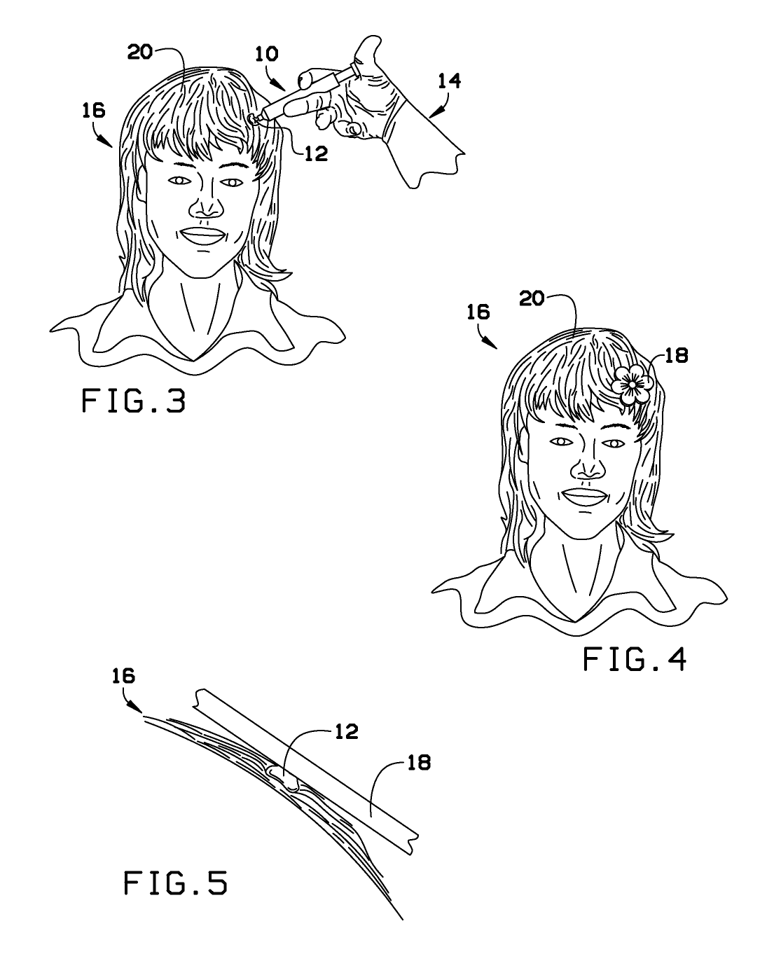

Referring now to FIGS. 3 through 5, an example of attaching an embellishment is shown. A user 16 desiring such an embellishment 18 can have another person 14 (or the user themselves) apply a water soluble hair glue 12, with the applicator 10, to a user's hair 20. The embellishment 18 can be applied to the warm, liquid water soluble hair glue 12 and, once cooled, the embellishment 18 is then attached to the hair 20. Washing the hair with shampoo and warm water can easily rinse out the water soluble hair glue 12 and remove the embellishment 18.

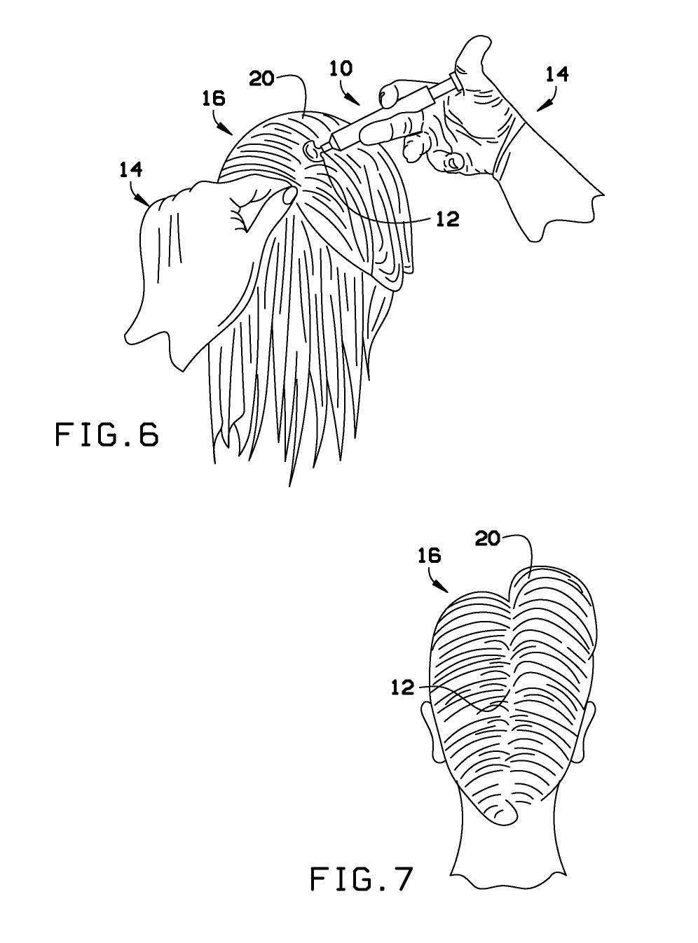

Referring now to FIGS. 6 and 7, the water soluble hair glue 12 can also be used to style one's hair. The water soluble hair glue 12 can be applied to the hair 20 and the hair 20 can be styled and held in place as desired. Again, simply washing the hair with shampoo and warm water can easily rinse out the water soluble hair glue 12 and allow the user to choose and create a new hair style.

While the above FIGS. 3 through 7 show use on a human's hair, the water soluble hair glue of the present invention can be used just as effectively on hair or fur of a pet, such as a cat or a dog, for example. The water soluble hair glue can be used in the same way as described above and can be removed by simply shampooing the pet when desired.

It should be understood, of course, that the foregoing relates to exemplary embodiments of the invention and that modifications may be made without departing from the spirit and scope of the invention as set forth in the following claims.

What is claimed is:

1. A water soluble hair glue comprising:
glycerin;
gelatin; and
water, wherein
the water soluble hair glue is a solid at room temperature and can be warmed to a liquid state for application to a user's hair.

2. The water soluble hair glue of claim 1, wherein the water soluble hair glue includes from about 17-23 parts by volume glycerin, from about 8-12 parts by volume gelatin, and from about 0.8 to about 1.5 parts water.

3. The water soluble hair glue of claim 1, wherein the water soluble hair glue includes about 20 parts by volume glycerin, about 10 parts by volume gelatin, and about 1.2 parts water.

4. A method for preparing a water soluble hair glue, comprising:
mixing glycerin and gelatin;
warming in a microwave oven for about 30 seconds;
allowing the mixture to cool;
warming in the microwave oven for about 40 seconds;
adding water to the resulting mixture while warm; and
allowing the resulting mixture to cool to a solid state.

5. The method of claim 4, further comprising warming the resulting solid state product to a liquid state and filling applicators with the liquid state product.

6. The method of claim 5, wherein the water soluble hair glue includes from about 17-23 parts by volume glycerin, from about 8-12 parts by volume gelatin, and from about 0.8 to about 1.5 parts water.

7. The method of claim 5, wherein the water soluble hair glue includes about 20 parts by volume glycerin, about 10 parts by volume gelatin, and about 1.2 parts water.

8. A method for styling a user's hair with either a hair style held in place by a water soluble hair glue or with an embellishment held in place with the water soluble hair glue, the method comprising:
warming a water soluble glue to a liquid state;
applying the water soluble glue to the user's hair, the water soluble hair glue including glycerin, gelatin, and water, wherein the water soluble hair glue is a solid at room temperature and can be warmed to a liquid state for application to a user's hair;
attaching a portion of the user's hair to the water soluble glue, or attaching the embellishment to the water soluble glue, while the water soluble glue is still in the liquid state; and
allowing the water soluble glue to cool to hold the portion of hair or the embellishment in place.

9. The method of claim 8, further comprising washing the user's hair to remove the water soluble hair glue therefrom.

10. The method of claim 8, wherein the water soluble hair glue includes from about 17-23 parts by volume glycerin, from about 8-12 parts by volume gelatin, and from about 0.8 to about 1.5 parts water.

11. The method of claim 8, wherein the water soluble hair glue includes about 20 parts by volume glycerin, about 10 parts by volume gelatin, and about 1.2 parts water.
